(19) United States
(12) Patent Application Publication
(10) Pub. No.: US 2022/0313634 A1
(43) Pub. Date: Oct. 6, 2022

(54) AGENT FOR THE PROPHYLACTIC AND/OR SUPPORTIVE THERAPEUTIC TREATMENT OF PARKINSON'S DISEASE

(71) Applicants: Horst Przuntek, Bochum (DE); Aiden Haghikia, Bochum (DE)

(72) Inventors: Horst Przuntek, Bochum (DE); Aiden Haghikia, Bochum (DE)

(21) Appl. No.: 17/807,797

(22) Filed: Jun. 20, 2022

Related U.S. Application Data
(63) Continuation of application No. 16/627,688, filed on Dec. 30, 2019, now abandoned.

(30) Foreign Application Priority Data
Jul. 13, 2017 (DE) 10 2017 115 819.7

(51) Int. Cl. A61K 31/19; A61P 25/16; A61K 9/48; A61K 33/00; A61K 33/06

(52) U.S. Cl. CPC A61K 31/19; A61P 25/16; A61K 9/48; A61K 33/00; A61K 33/06

(57) ABSTRACT

The invention relates to an agent for use in the prophylactic and/or supportive therapeutic treatment of Parkinson's disease, said agent comprising a physiologically effective amount of propionic acid and/or butyric acid and/or physiologically compatible salts or esters thereof.

The present invention relates to an agent for the prophylactic and/or supportive therapeutic treatment of Parkinson's disease.

Numerous illnesses are associated with a deficient colonization of the intestine, that is the microbiome of the intestine is out of balance, whether due to a deficient colonization or the loss of essential constituents for the maintenance of bodily functions. In the case of all these diseases, such as obesity, ulcerative colitis, multiple sclerosis (MS), Parkinson's disease and presumably also psychoses, but also in rheumatic disease forms and psoriasis, significant changes in the microbiome of the intestine can be detected. This suggests that microbiome and the respective disease are interlinked, either as a result of a common cause or due to reciprocally acting influencing factors.

It has further been found that the intestinal microbiome can be influenced by the type of nutrition consumed and is capable of adapting to the requirements to be met for a given kind of food. This means, an unfavorable intestinal microbiota unfavorable for the immune status of the patient can be changed by taking suitable dietary measures aimed at improving the immune status of the patient.

The invention is based on the findings that in Parkinson's disease a characteristic change in the microbiome has occurred compared to healthy control subjects. In particular, bacteria producing short-chain fatty acids are hardly or not at all present in the microbiome. Accordingly, there is a lack of short-chain fatty acids, which occur in the intestines of a healthy control person as degradation products. Braak has postulated that Parkinson's disease begins in the intestine. This is in line with the observation that the transfer of the microbiome from mice with Parkinson's disease to healthy mice produces a comparable picture of Parkinson's disease.

The lack of microorganisms producing short-chain fatty acids in Parkinson's patients causes, in particular, a lack of acetic, propionic and butyric acid. While acetic acid is supplied abundantly with food, the deficiency of propionic and butyric acid is usually not compensated.

Alpha-synuclein, a transport protein that occurs in the brain and plays a role in Parkinson's disease, can be detected in very early stages of the disease in the intestine and serves as an indicator of the disease. This also suggests a connection between the disease and what happens in the intestine.

Experiments have been conducted to compensate for deficits in intestinal colonization by taking medicinal or dietary measures, but this has only been successful to a limited extent.

Surprisingly, it has been found that propionic acid and butyric acid have a positive effect on the development and course of Parkinson's disease. This also applies to their physiologically tolerable salts and esters. It was also observed that the targeted administration of these substances improves the medication treatment of Parkinson's disease, i.e. has an intensifying effect. In particular, the dosage of dopaminergic drugs, which are usually employed in the treatment of Parkinson's disease, can be significantly reduced.

Accordingly, the invention relates to an agent for use in the prophylactic and/or supportive therapeutic treatment of Parkinson's disease, with said agent comprising a physiologically effective amount of propionic acid and/or butyric acid and/or physiologically acceptable salts or esters thereof.

The agent proposed by the present invention may be administered both for prophylactic and therapeutic purposes to persons with a predisposition to Parkinson's disease or to Parkinson's patients. However, the agent is in particular suitable for the supportive therapeutic treatment of Parkinson's patients, who otherwise undergo conventional medication treatment.

The inventive agent may contain propionic acid or butyric acid alone or in combination thereof. However, administration in the form of physiologically acceptable salts is preferred, with the salts of physiologically important metals being to the fore. Aside from alkali and alkaline earth salts, these may also include zinc and iron salts.

Particularly preferred are the salts of sodium, potassium, magnesium and calcium of both propionic acid and butyric acid.

In addition, propionic acid and butyric acid can also be administered in the form of their esters. Esters of C₁ to C₆ alcohols, in particular methyl and ethyl esters, are particularly suitable. The esters are hydrolyzed in the body to free acids.

The inventive agent may be administered in usual forms, for example, as tablets, dragées, pills, capsules, lozenges, powders and granules. An administration in liquid form is also possible in the form of juices, drops and teas. In any case, the agent is intended for oral administration.

Preferred agent delivery forms are tablets, capsules and powder. The tablets and capsules containing a unit dose of the inventive agent are preferably administered twice daily. The powder can, for example, be stirred into a drink/beverage or added to food.

A unit dose for the aforementioned agent delivery forms is in the range of between 0.2 and 5 g, in particular between 0.3 and 3 g. A particularly preferred amount for tablets, capsules and powders is 0.5 to 2.0 g. intended for morning and evening administration each, and, where appropriate, additionally at midday, in particular in connection with meals.

Particularly preferred is the combined administration of propionic and butyric acid or salts and esters thereof in a single dose, but also in separate form. For example, the weight ratio in this case can be in the range from 3:1 to 1:3, in particular 3:2 to 2:3, for the total doses indicated above.

As already mentioned, the agent proposed by the invention can be employed for the supportive therapeutic treatment of Parkinson's patients. In this case, it is administered in addition to the usual medication treatment, for example together with levodopa and other dopaminergic drugs as they are commonly used. The dosage of the inventive agent is as indicated above.

The effect of butyric acid/butyrate was investigated in patient studies in which more than 1,000 test subjects have participated for at least one year each. A total amount of 6 g butyric acid or butyrate (as salt) was administered together with the physician-directed medication (levodopa etc.) in three daily doses with meals in the morning, at noon and in the evening. Significant improvements of the general condition were determined, in particular with respect to the motor functions.

Surprisingly it was discovered that the amount of dopamine used by Parkinson's patients treated with butyric acid/butyrate was reduced by at least 50%, in some cases by up to 90%. This is especially important because the usual medication for Parkinson's patients, not only with levodopa, can lead to considerable side effects such as dizziness, nausea, tachyarrhythmia, psychosis, dyskinesia and circulatory problems.

Finally, the invention relates to a dietary supplement containing propionic acid and/or butyric acid, their physiologically acceptable salts or esters, alone or in a mixture. Preferred for this purpose is butyric acid or a butyrate, where considered appropriate together with propionic acid or a propionate, particularly in the form of capsules or tablets.

The dietary supplement in capsule or tablet form preferably contains propionic acid and butyric acid in the form of a salt each. The weight ratio in this case is in particular 3:1 to 1:3 for a total amount of 0.5 to 2.0 g.

1. Agent for use in the prophylactic and/or supportive therapeutic treatment of Parkinson's disease, said agent comprising a physiologically effective amount of propionic acid and/or butyric acid and/or physiologically acceptable salts or esters thereof.

2. Agent according to claim 1, characterized in that the salts of the propionic acid and/or butyric acid are the alkali or alkaline earth salts.

3. Agent according to claim 2, characterized in that the salts are sodium, potassium, magnesium or calcium salts.

4. Agent according to claim 1, characterized in that the esters are methyl or ethyl esters.

5. Agent according to claim 1 in tablet, capsule or powder form.

6. Agent according to claim 1, characterized in that said agent is made up in individual doses of 0.2 to 5 g.

7. Agent according to claim 6, characterized in that an individual dose contains 0.5 to 2.0 g of active substance, if appropriate together with customary preparation agents and auxiliary substances.

8. Agent according to claim 1, containing a combination of propionic acid and butyric acid or the salts or esters thereof.

9. Agent according to claim 8, containing propionic acid and butyric acid or salts or esters thereof in a weight ratio of 25/75 to 75/25.

10. Use of propionic acid and butyric acid or salts and esters thereof for the preparation of an agent for the prophylaxis and/or supportive treatment of Parkinson's disease.

11. Dietary supplement containing propionic acid and butyric acid and/or their physiologically acceptable salts or esters.

12. Dietary supplement according to claim 11, characterized in that said supplement contains propionic acid and butyric acid in the form of their physiologically compatible salts, in particular their sodium and/or calcium salts.

13. Dietary supplement according to claim 11 in capsule form.
